(19) United States
(12) Patent Application Publication
(10) Pub. No.: US 2011/0129864 A1
(43) Pub. Date: Jun. 2, 2011

(54) METHOD FOR ANALYZING LYMPH NODE ASPIRATE USING MULTI-ANGLE LIGHT SCATTER FLOW CYTOMETER

(75) Inventors: Melissa Jane Beall, Cape Elizabeth, ME (US); James W. Russell, North Yarmouth, ME (US)

(73) Assignee: IDEXX LABORATORIES, INC., Westbrook, ME (US)

(21) Appl. No.: 12/629,202

(22) Filed: Dec. 2, 2009

(51) Int. Cl. C12Q 1/02

(52) U.S. Cl. 435/29

(57) ABSTRACT

Disclosed is a method for identifying lymphatic disease and disease states in mammals. The method uses a multi-angle light scatter flow cytometer to diagnose and treat mammals. The method includes collecting lymph node aspirate from a mammal; scanning the lymph node aspirate in a flow cytometer to generate a diagnostic scan; comparing the diagnostic scan to a known normal scan; identifying differences between the diagnostic scan and the known normal scan; and identifying similarities between the diagnostic scan and known disease scans to identify a cause of lymphadenopathy. Graphical representations of leukocyte identification are generated as a result of the scanning process. By using the graphs, a veterinarian or technician is able to diagnose the effectiveness or ineffectiveness of the treatment.

### BACKGROUND

1. Technical Field

The present disclosure relates to the use of flow cytometry in the diagnosis and treatment of mammals. More specifically, the disclosure relates to methods of identifying lymphatic disease and disease states in mammals.

2. Background of Related Art

In a healthy animal, the lymphoid system is an important part of the body's immune system defense against infectious agents such as viruses and bacteria. Lymphoid tissue is normally found in many different parts of the body including lymph nodes, liver, spleen, gastrointestinal tract and skin. Lymphadenopathy in mammals is often indicative of infection or inflammation. However, lymphadenopathy may also be caused by more serious conditions such as, for example, leukemia, lymphoma, or metastatic tumors.

Lymphoma is one of the most common cancers seen in dogs. Although there are breeds that appear to be at increased risk for this disease, lymphoma can affect any dog of any breed, at any age. Lymphoma accounts for 10-20% of all cancers in dogs. Lymphoma (lymphosarcoma or non-Hodgkin's lymphoma) is a malignant cancer that involves the lymphoid system. Lymphoma is classified according to the location in the body in which the cancer begins. For example, multicentric lymphoma occurs in the lymph nodes while gastrointestinal lymphoma occurs in the stomach, intestines, liver, and abdominal lymph nodes.

Treatments for dogs with cancer, much like those for humans, may take the form of conventional (chemotherapy, surgery, radiation therapy, etc.), alternative (holistic, herbal, etc.), or complementary. Identifying the cause of lymphadenopathy, especially persistent lymphadenopathy, often requires aspiration of lymphoid cells from the abnormal lymph node. This aspirate is then placed on a slide with a cover slip for evaluation by a cytologist or clinical pathologist. The process often ruptures a number of cells which cannot be evaluated.

Newer techniques in the evaluation of lymphoproliferative diseases have involved fluorescent flow cytometry. However, these methods utilize whole blood samples which require extensive processing and labeling. The processing and labeling of whole blood cell samples in order to identify and evaluate lymphoproliferative diseases poses additional challenges, such as purification of whole blood samples to obtain lymphocytes exclusively. Furthermore, the lymphocytes collected from whole blood samples often include a lower population of reactive cells, i.e., cells indicative of the specific disease state. Moreover, the cost and extensive laboratory preparation involved with preparing a fluorescent marker is undesirable. In the absence of a fluorescent marker, buffer solutions for sample scanning may be prepared at the clinic instead of the laboratory.

Accordingly, improved methods of identifying the cause of lymphadenopathy in a mammal and monitoring any disease causing the lymphadenopathy, are still needed.

### SUMMARY

The following embodiments and aspects thereof are described and illustrated in conjunction with systems, tools and methods which are meant to be exemplary and illustrative, not limiting in scope.

It is therefore an object of the disclosure to identify lymphatic disease and disease states in mammals. The present disclosure relates to a method for identifying a cause of lymphadenopathy in a mammal. The method includes (a) collecting lymph node aspirate from a mammal; (b) scanning the lymph node aspirate in a flow cytometer to generate a diagnostic scan; (c) comparing the diagnostic scan to a known normal scan; (d) identifying differences between the diagnostic scan and the known normal scan; and (e) identifying similarities between the diagnostic scan and known disease scans to identify a cause of lymphadenopathy.

A further aspect of the present disclosure includes a method for monitoring a disease state in a diseased mammal. The method includes (a) aspirating a lymph node of a diseased mammal to obtain pre-treatment cells; (b) scanning the pre-treatment cells using a multi-angle scattered flow cytometer to generate a first scatter profile; (c) aspirating a lymph node of a diseased mammal following treatment to obtain post-treatment cells; (d) scanning the post-treatment cells using the multi-angle scattered flow cytometer to generate a second scatter profile; and (e) identifying differences between the first scatter profile and the second scatter profile.

The present disclosure also includes a method for of assessing a disease state including collecting lymph node aspirate from a mammal; scanning the lymph node aspirate in a flow cytometer to generate a first disease state scan; collecting an additional lymph node aspirate from the mammal; scanning the additional lymph node aspirate in a flow cytometer to generate an additional disease state scan; and comparing the first disease state scan to the additional disease state to assess the disease state.

### BRIEF DESCRIPTION OF THE DRAWINGS

These and other objects and features of the disclosure will become more fully apparent when the following detailed description of the disclosure is read in conjunction with the accompanying drawings.

FIG. 1 is an exemplary schematic representation of a multi-angle flow cytometer in accordance with an embodiment of the present disclosure;

FIG. 2 is an exemplary schematic representation of the electro-optical components in accordance with an embodiment of the present disclosure;

FIG. 3 is an exemplary block diagram of the electronic processing components in accordance with an embodiment of the present disclosure;

FIG. 4 is an exemplary flow diagram of a method performed in accordance with one illustrative embodiment of the present disclosure;

FIG. 5 is another exemplary flow diagram of a method performed in accordance with another illustrative embodiment of the present disclosure;

FIG. 6 is an exemplary scatter profile defined by taking a normal lymph node aspirate sample in accordance with an illustrative embodiment of the present disclosure;

FIG. 7 is an exemplary scatter profile defined scanning lymph node aspirate from a newly diagnosed lymphoma patient prior to treatment in accordance with an illustrative embodiment of the present disclosure;

FIG. 8 is an exemplary scatter profile defined by scanning lymph node aspirate from a lymphoma patient receiving treatment in accordance with an illustrative embodiment of the present disclosure;

FIG. 9 is an exemplary scatter profile defined by scanning lymph node aspirate from a patient with reactive inflammation in accordance with an illustrative embodiment of the present disclosure; and

FIG. 10 is an exemplary scatter profile defined by scanning lymph node aspirate from a patient with a metastatic mast cell tumor as sample in accordance with an illustrative embodiment of the present disclosure.

### DETAILED DESCRIPTION

The present disclosure is directed to methods for identifying a cause of lymphadenopathy in a mammal, as well as determining the impact of a given therapeutic technique on an active disease state.

As used herein, the term “disease” or “diseased” relate primarily to lymphadenopathy and lymphoproliferative diseases. Lymphoproliferative diseases include, but are not limited to, lymphocytic leukemia, lymphoma, and metastatic mast cell tumors.

As used herein, the term “disease state” relates to the presence and level of activity of disease in a mammal. Accordingly, an active “disease state” would indicate that the disease is present and at an increasing level.

As used herein, the term “cause of lymphadenopathy” may refer to inflammation, disease, and/or infection of the lymph nodes;

As used herein, the term “mammal” includes all mammals. In some embodiments the mammal is canine. In embodiments, the mammal is feline.

As used herein, the term “treatment” means a therapy directed at reducing the overall disease state. Treatment may include, for example, pharmaceutical medicaments, so called “over-the-counter” treatments and/or supplements, “non-traditional” treatments such as acupuncture and chiropractic methods.

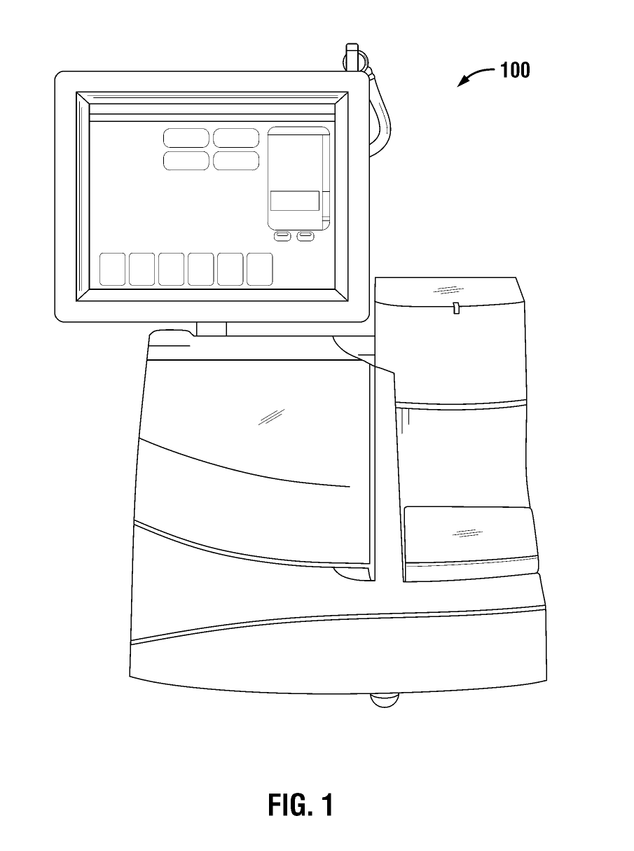

FIG. 1 is an example of a multi-angle flow cytometer 100. Flow cytometers have been commercialized and are known in the art. IDEXX Laboratories has developed a commercial flow cytometer for analysis of blood which is marketed under the trademark LASERCYTE™. Flow cytometers are also described in the patent literature, see for example U.S. Pat. Nos. 6,784,981 and 6,618,143, both assigned to IDEXX Laboratories, the contents of which are incorporated by reference herein in their entirety.

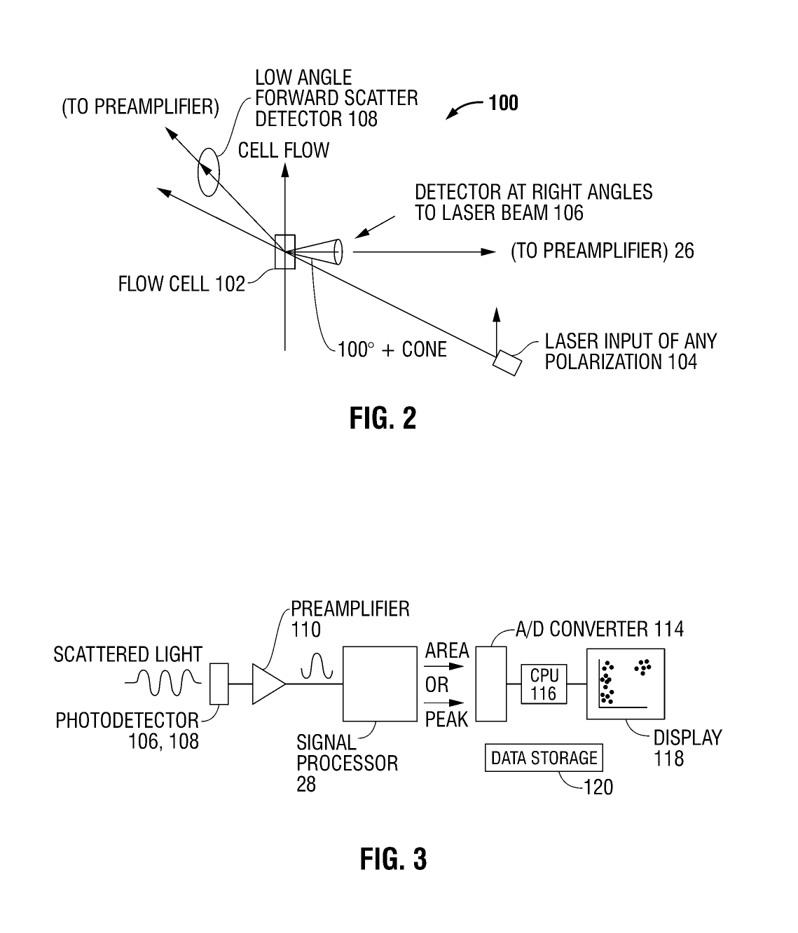

The flow cytometer 100 may be a hematology analyzer for veterinary diagnostics at point of care veterinary clinics. This type of flow cytometry 100 may use a high numerical aperture flow cytometer. As shown in FIG. 2, the flow cytometer 100 may include a flow cell 102 through which cells derived from lymph node aspirate may be flowed. Laser input 104 emits a beam of light that is oriented substantially orthogonally to the flow of cells through the flow cell 102. A portion of the beam from laser input 104 that impinges upon the cells in flow cell 102 is scattered at a substantially right angle to the beam of laser input 104 (right angle scattered light). A second portion of the beam from laser input 104 that impinges upon the cells in flow cell 102 is scattered at a much lower angle than 90°. This scatter is termed “low angle forward scattered light” (FSL) and has an angle of from approximately 1° to approximately 3° from the orientation of the original beam from laser input 104. Right angle scatter light detector 106 is oriented to receive the previously mentioned right angle scattered light. In some embodiments, right angle scatter light detector 106 is located about 2 millimeters from the cells in the flow cell 102. At the distance of about 2 millimeters from the cells, right angle scatter light detector 106 collects a cone of scattered light of at least 100° or greater. In some embodiments, right angle scatter light detector 106 collects a cone of scattered light of at least 130° or greater. This larger light cone results in the greater cluster separation. The low angle forward scatter light detector 108 is oriented to capture the previously mentioned low angle forward scatter light oriented at approximately 1° to approximately 3° from the beam of the laser input 104. In the flow cytometer 100, the light signals may include: extinction (EXT) (0°-approximately 0.5°); low angle forward scattered light (FSL) (approximately 1°-approximately 3°); high angle forward scattered light (FSH) (approximately 4°-approximately 9°); and side scattered light (SS) (approximately 50°-approximately 130°). Time-of-flight (TOF) measurements may also be made.

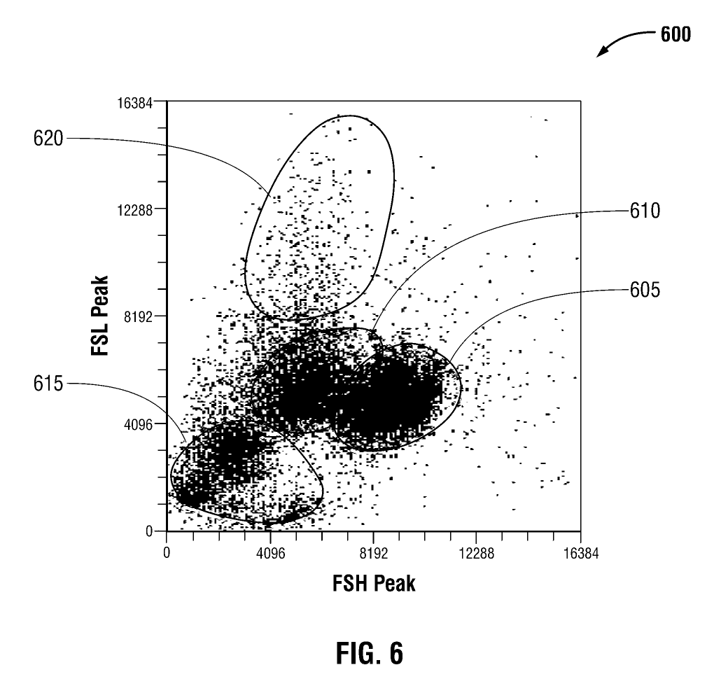

As shown in FIG. 3, the electrical outputs from right angle scatter light detector 106 and low angle forward scatter device 108, which may be in voltage or current form, for example, are amplified by preamplifier 110 and then sent to signal processor 112. Signal processor 112 measures the area under the voltage or current curve, or measures the peak of the voltage or current curve, received from right angle light scatter detector 106 and/or low angle forward scatter light detector 108. The data from signal processor 112 is converted by analog to digital converter 114. The digital data is next processed by central processing unit 116 based on software programs to display the data in graphical representation on display 118. Such a device is described, for example, in U.S. Pat. No. 6,320,656, incorporated herein by reference, in its entirety. The flow cytometer 100 may also include data storage 120. Data storage 120 may be a volatile memory or a nonvolatile memory. Data storage 120 may generally be any type of memory used in a single processor architecture including random access memory (RAM) or read only memory (ROM). Scatter profiles such as are shown in FIGS. 6-10 may be stored and retrieved from the data storage 120 for comparison purposes to determine the effectiveness or ineffectiveness of treatment as further explained below.

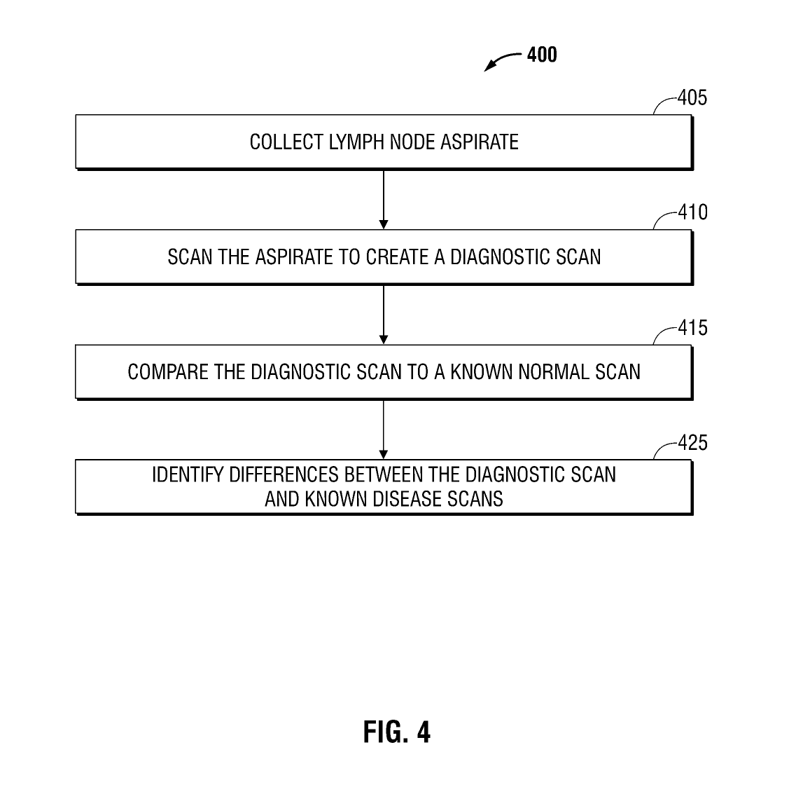

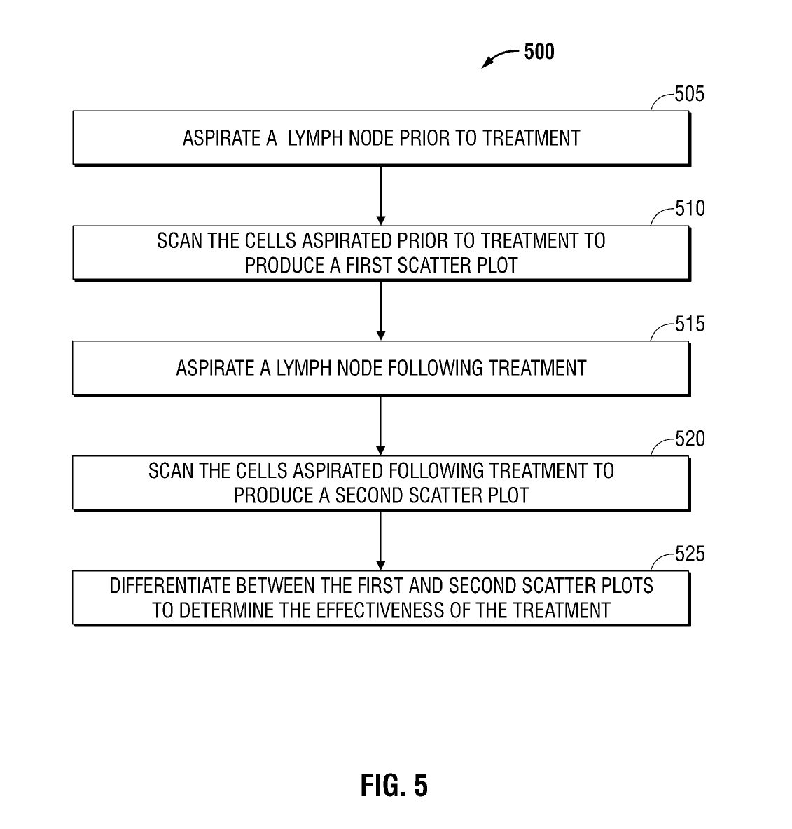

As further discussed below with references to FIGS. 4 and 5, a veterinarian or technician may be able to diagnose the source of lymphadenopathy in a mammal using the method disclosed. Referring to flowchart 400 of FIG. 4, an embodiment of a method for identifying a cause of lymphadenopathy in a mammal is disclosed. The method includes collecting lymph node aspirate from a mammal (step 405). The mammal may include canines, felines, humans, apes, bats, tigers, mice, moose, elephants, gorillas, sloths, pandas, hamsters, horses, whales, dolphins, and other types of mammals.

In accordance with the present disclosure, a lymph node aspirate is scanned on a flow cytometer. Lymph nodes function to trap foreign particles. Lymph nodes contain a fluid known as lymph which is similar to plasma. They also contain a high number and variety of subpopulations of white blood cells. White blood cells in the lymph nodes may be exposed to the foreign particles and may then mount a defense to any foreign invading particle such as a virus or bacteria. The concentration and variety of white blood cells aspirated from the lymph node is much higher than the quantity in peripheral blood. Additionally, lymph node aspirate does not typically contain a large number of red blood cells relative to the number of lymphocytes, thereby removing the need for lysing red blood cells prior to scanning. Accordingly, lymph node aspirate may provide a detailed profile of activity within the immune system.

As stated above, white blood cells and leukocytes are the immune system cells that destroy foreign agents, such as bacteria, viruses, and other pathogens that cause infection. WBC concentrations exist in peripheral blood in very low concentrations as compared to their concentration in lymph node aspirate. There are a variety of white blood cell types that perform different functions within the body. In this application, the terms “white blood cells,” “white cells,” “leukocytes,” and “WBCs” are used interchangeably to refer to the non-hemoglobin-containing nucleated blood cells present in the circulation. WBCs typically have diameters between 6 and 13 microns, depending on the subpopulation of white blood cells and the species.

Granular white blood cells, or granulocytes, may be further subdivided into neutrophils, eosinophils, and basophils. The most prevalent of the granulocytes are the neutrophils. They typically have a diameter of about 12 μm.

Agranular white blood cells are sometimes referred to as mononuclear cells, and are further sub-classified as either lymphocytes or monocytes. Lymphocytes are the most prevalent of the mononuclear cell types, and generally make up between 20 and 30 percent of the total number of WBCs and are about 7-9 μm in diameter. Lymphocytes specifically recognize foreign antigens and, in response, divide and differentiate to form effector cells. The effector cells may be B lymphocytes or T lymphocytes. B lymphocytes secrete large amounts of antibodies in response to foreign antigens. T lymphocytes exist in two main forms—cytotoxic T cells, which destroy host cells infected by infectious agents, such as viruses; and helper T cells, which stimulate antibody synthesis and macrophage activation by releasing cytokines. Many lymphocytes differentiate into memory B or T cells, which are relatively long-lived and respond more quickly to foreign antigen than naïve B or T cells.

Monocytes are immature forms of macrophages that, in themselves have little ability to fight infectious agents in the circulating blood. However, when there is an infection in the tissues surrounding a blood vessel, these cells leave the circulating blood and enter the surrounding tissues. The monocytes then undergo a dramatic morphological transformation to form macrophages, increasing their diameter as much as fivefold and developing large numbers of mitochondria and lysosomes in their cytoplasm. The macrophages then attack the invading foreign objects by phagocytosis and activation of other immune system cells, such as T cells. Increased numbers of macrophages are a signal that inflammation is occurring in the body.

Platelets are found in all mammalian species, and are involved in blood clotting. These cellular particles are usually very small, having a diameter between 1 and 3 μm. “Platelet aggregates” as used herein, refer to two or more clumped platelets and large platelets, i.e., platelets greater than 4 μm in diameter.

As disclosed above, flow cytometry may be used to identify and enumerate white blood cell subpopulations and determine disease status based on these results. White blood cells in a buffer solution are caused to flow individually through a light beam, produced by a laser light source. As light strikes each cell, the light is scattered and the resulting scattered light is analyzed to determine the type of cell.

Different types of cells produce different types of scattered light. The type of scattered light produced may depend on the degree of granularity, the size of the cell, etc.

According to the present disclosure, a method for identifying a cause of lymphadenopathy is provided. One illustrative embodiment is for the detection of canine lymphoma. A method for monitoring a disease state, i.e., determining severity or remission of disease, based on white blood cell subpopulations from lymph node aspirate before, during and/or following treatment is provided. This may allow for determination of the effect of a particular treatment on a mammal. A method for assessing disease state in a mammal is also provided. This may allow for the monitoring of progression of a disease over time. Although the present disclosure will primarily address the identifying and determining severity or remission of disease states as relates to canine lymph node aspirate, it is clearly not limited thereto.

Lymph node aspirate of a canine may be prepared as follows, prior to analysis on flow cytometer 100. The lymph node aspirate may be diluted 1 to 10 in a suitable buffer, such as, phosphate buffered saline without the use of a marker, such as, for example, a fluorescent agent. Variations of the above preparation method, such as are known to those of skill in the art, may be employed as necessary.

The prepared solution is then placed in the flow cytometer 100. With continued reference to FIGS. 1 and 4, the flow cytometer 100 scans the diluted aspirate to generate a diagnostic scan (step 410). The diagnostic scan may represent, for example, the current condition of a canine with lymphadenopathy. The scanning may include scanning the aspirate in the absence of a marker.

The method may further include comparing the diagnostic scan to a known normal scan (step 415). The known normal scan may be a scan from lymph node aspirate of a healthy mammal of the same or a similar species. The diagnostic and known normal scans are scatter profiles of a peak of the FSL versus a peak of the FSH. An identification of disease, or a cause of lymphadenopathy, may be performed by analyzing the differences between the diagnostic scan and the known normal scan (step 420). The method may further include identifying similarities between the diagnostic scan and scans of aspirate from mammals with a known disease (known disease scans). This may be used to identify a particular cause of lymphadenopathy (step 425). The known disease scans may also be a scatter profile of a peak of the FSL versus a peak of the FSH. The known disease scans may include, for example, a scan of lymph node aspirate from, for example, a mammal with lymphoma, a metastatic mast cell tumor, or pyogranulomatous inflammation. In the case of either known normal scans or known disease scans, the scatter profiles associated with these scans can be stored in the analyzer's memory for future comparison to patient samples.

Identification of the differences and similarities between the diagnostic, known normal, and known disease scans, may be performed with the naked eye or using a computer program for analysis. As described in greater detail below, scans that are diagnostic for a disease state may exhibit a characteristic pattern. This pattern is based on the number of lymphocyte subpopulations present in the extracted lymph. A semi-quantitative analysis of each lymphocyte subpopulation identified in the scan may be performed on two scans of the same patient. This information may then be used to, for example, monitor a disease state, or determine the effect of a treatment. Software and algorithms designed to perform this type of analysis may also be used to perform a semi-quantitative analysis.

There are a wide variety of chemotherapy protocols and drugs currently used to treat lymphoma. Treatment usually consists of a combination of oral and injectable drugs given on a weekly basis. Some commonly used drugs include cyclophosphamide, vincristine, doxorubicin, and prednisone. The exact treatment protocol varies depending on a number of factors including, for example, disease state, age and weight of the mammal, and the treating veterinarian.

When administering chemotherapeutic treatments to mammals, other than humans, discussing treatment effectiveness cannot typically involve a verbal consultation. Accordingly, the present disclosure further includes a method to determine the level of effectiveness or ineffectiveness of a treatment regimen. Following implementation of a treatment regimen, a certain period of time, dependent on the regime, may need to pass before laboratory results reflect the effect of the treatment. For example, it may be useful to repeat the above steps on a weekly, biweekly, or monthly basis, depending on, for example, the treatment regime and the disease, to identify the effectiveness or ineffectiveness of treatment. Once sufficient time has passed for the impact of the regime to be reflected, the following steps may be repeated to determine the effect of the treatment: (a) collecting lymph node aspirate from the mammal to obtain additional treatment aspirate; (b) scanning the additional treatment aspirate in the flow cytometer to generate an additional diagnostic scan; (c) comparing the additional diagnostic scan to the known normal scan; (d) identifying differences between the additional diagnostic scan and the known normal scan; and (e) identifying similarities between the another diagnostic scan and the known disease scans to identify another particular disease state.

Referring to flowchart 500 of FIG. 5, an embodiment for a method of monitoring a diseased mammal is disclosed. The method may include aspirating cells from a lymph node of a diseased mammal prior to treatment (step 505). The method may further include scanning the aspirate taken prior to treatment using a multi-angle scattered flow cytometer to generate a first scatter profile (step 510). The first scatter profile may include subpopulations and patterns of the cells aspirated prior to treatment. As further explained below with references to FIGS. 6-10, the subpopulations and patterns may include clusters of white blood cell subpopulations. By analyzing the scatter profiles with subpopulations and patterns, a veterinarian or technician, for example, is able to determine the effectiveness or ineffectiveness of a particular treatment.

A lymph node of a diseased mammal may be aspirated following treatment (step 515). The cells aspirated following treatment may be scanned to generate a second scatter profile using the flow cytometer (step 520). The second scatter profile will include subpopulations and patterns of the cells aspirated following treatment. The subpopulations and patterns of the first and second scatter profiles may be generated using high forward scattered light and low forward scattered light techniques.

The method may further include identifying differences in the subpopulations and patterns of the first and second scatter profiles to determine the effectiveness of the treatment (step 525). The differentiation between the subpopulations and patterns of the first and second scatter profiles may include determining for each of the first and second scatter profiles, a ratio of healthy lymphocytes versus malignant lymphocytes, which may be used to determine the effectiveness of the treatment. Moreover, the first and second scatter profiles of clusters may represent a size of the lymphocytes.

In another embodiment, the method 500 may further include scanning dilute lymph node aspirate taken prior to treatment in the flow cytometer. After producing a first scatter profile, the method may further include scanning dilute lymph node aspirate taken following treatment to generate a second scatter profile. The method 500 may further include comparing specific lymphocyte population levels as described in FIGS. 6-9 below to the first scatter profile and/or to a baseline scatter profile. A baseline scatter profile may be derived from lymph node aspirate of a healthy mammal.

In another embodiment, it may be useful to repeat the method 500 for further analysis. The repeated method may include (a) aspirating lymph cells of a diseased mammal following an additional treatment; (b) scanning the aspirated cells using a multi-angle scattered flow cytometer to generate an additional scatter profile, wherein the additional scatter profile includes subpopulations and patterns of the cells aspirated following the additional treatment; and (c) differentiating between the subpopulations and patterns of the first, second, and additional scatter profiles to determine the effectiveness of the treatment. The additional treatment may be different or the same as the original treatment. For example, the original treatment may be chemotherapy followed by herbal treatment. A veterinarian or technician may be able to analyze the first, second, and additional scatter profiles to determine the effectiveness of the chemotherapy and herbal treatments.

A method of assessing disease state in a mammal is also provided. This method involves collecting lymph node aspirate from a diseased mammal. This aspirate is then scanned to produce a first diagnostic scan. This first diagnostic scan may include subpopulations of lymphocytes. These subpopulations may include, for example, normal lymphocytes, abnormal lymphocytes, granulocytes, monocytes, metastatic mast cells, T-cells, B-cells and combinations of these cells. As a disease progresses or remits in a mammal, additional scans may be taken. These additional scans require collecting an additional lymph node aspirate and scanning the additional lymph node aspirate to obtain an additional diagnostic scan of the same mammal. The first and additional diagnostic scan may be compared. The comparison may involve quantification of the lymphocyte subpopulations. The comparison may also involve comparing the quantity of normal lymphocytes in the first and additional scans. If the number of normal lymphocytes is increasing, this may indicate a remission of disease, however, if other subpopulations of lymphocytes are increasing the disease may be progressing. The rate of disease progression over time may also be monitored in this manner. The subpopulation of lymphocytes increasing, decreasing, or remaining the same in additional scans over time may indicate disease state.

Next, referring to FIGS. 6-10, graphical representations of lymphocyte subpopulation identification are shown. Regions 605, 705, 805, 905, and 1005 may occupy the same regions on different scatter profiles; the same is true for the other like numbered regions of the scatter profiles. The data of FIGS. 6-10 may be employed using the apparatus substantially disclosed in FIGS. 1-3 and more specifically, flow cytometers, for example U.S. Pat. Nos. 6,784,981 and 6,618,143, both assigned to IDEXX Laboratories, the contents of which are incorporated by reference herein in their entirety. A correlation exists between scans of lymph node aspirate of the present disclosure of FIGS. 6-10 and certain disease states in mammals. FIG. 6 is a scatter profile 600 of peak FSL versus peak FSH. Scatter profile 600 is for a normal healthy mammal, such as a dog. Scatter profile 600 has four regions 605, 610, 615, and 620. A first region 605 is a dark cloud just above FSH peak of about 8192 and FSL peak of about 4096. The first region 605 shows red blood cells. To the left of the first region 605, with an FSH at about 6000 peak and an FSL peak of about 5000 is a second dark region 610. The second dark region is normal lymphocytes. The third region 615, with an FSH peak of about 2000 and an FSL peak of about 2000 is a non-intact cells or debris region. The scatter along the baseline is also cell debris. The fourth region 620 is located at about 6000 FSH peak and about 8192 FSL and above, has very few abnormal and enlarged lymphocytes.

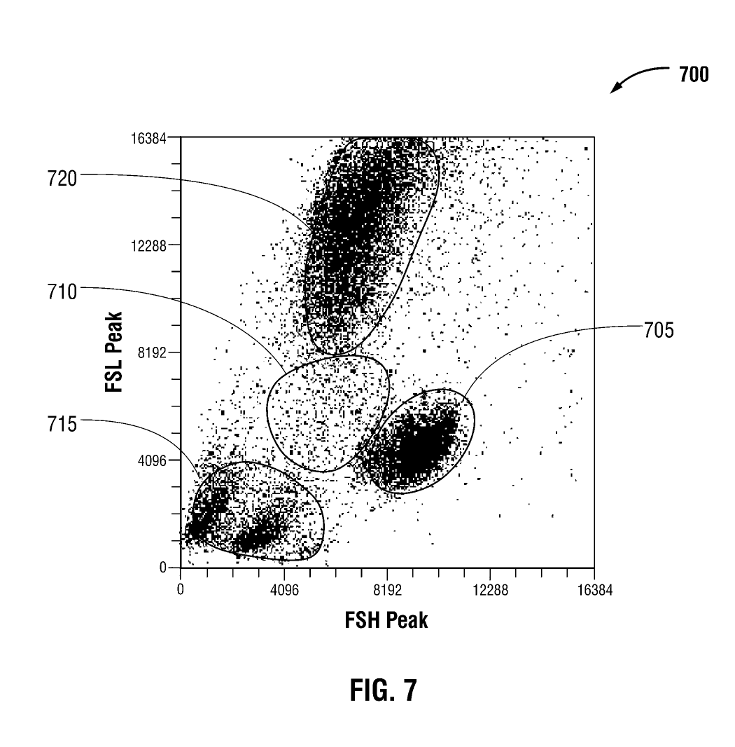

FIG. 7 is a scatter profile 700 of a newly diagnosed lymphoma mammal. Scatter profile 700 also has a first region 705 (about 9000 FSH peak and 4096 FSL peak) of red blood cells, second region 710 (about 5000 FSH peak and 5000 FSL peak) of normal lymphocytes, and third region 720 (about FSH peak of 6000 and FSL peak of about 8192 and above) of abnormal or enlarged lymphocytes. The fourth region 715 (FSL peak below 4096 and FSH peak below 5000) includes non-intact cells and debris. However, the third region 720 of abnormal and enlarged lymphocytes is pillar-like in shape and much more densely populated than region 620 of FIG. 6. There are far fewer normal lymphocytes in region 710, as compared to the region 610 of FIG. 6, since the majority is now represented by the abnormal cells in region 720.

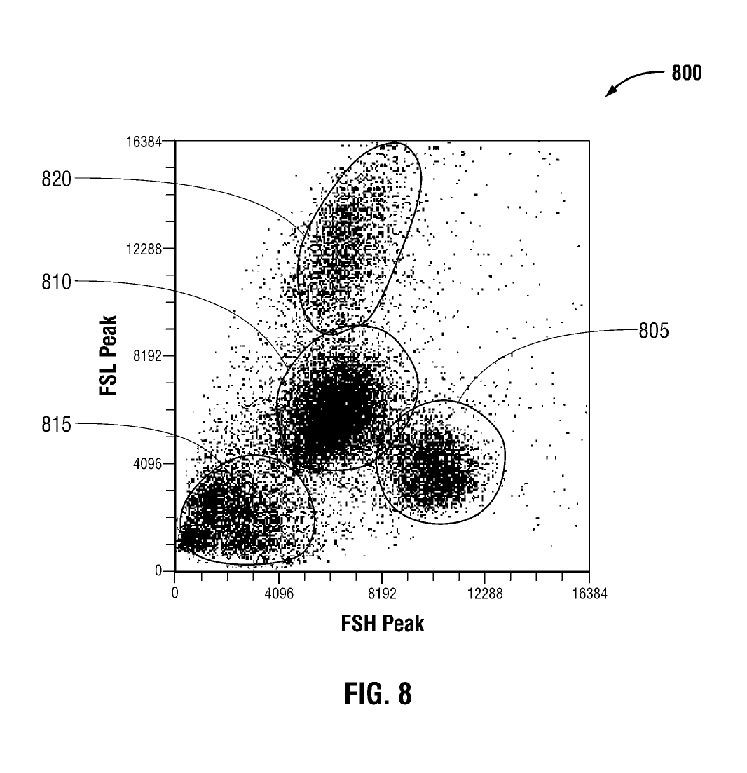

FIG. 8 is a scatter profile 800 of a mammal after treatment such as chemotherapy. Similar to FIGS. 6 and 7, scatter profile 800 has a first region 805 of red blood cells. The second region 810 of normal lymphocytes is visible and more densely populated than region 710 in scatter profile 700 (FIG. 7). The non-intact cell and debris region is represented by 815. The region 820 which represents the enlarged and abnormal lymphocytes is still retaining some of the pillar effect, but is reduced in density due to an increase in the numbers of normal lymphocytes in region 810. A comparison of the regions 810 and 820 of FIG. 8 and the regions 710 and 720 of FIG. 7, a veterinarian or technician may be able to draw a conclusion that the current treatment regimen is having a positive effect.

Due to fragility of the abnormal lymphocytes, red cell lysis prior to scanning is typically not performed. Red cell contamination may occur with sampling of lymph nodes by aspiration, but may vary with each sampling event.

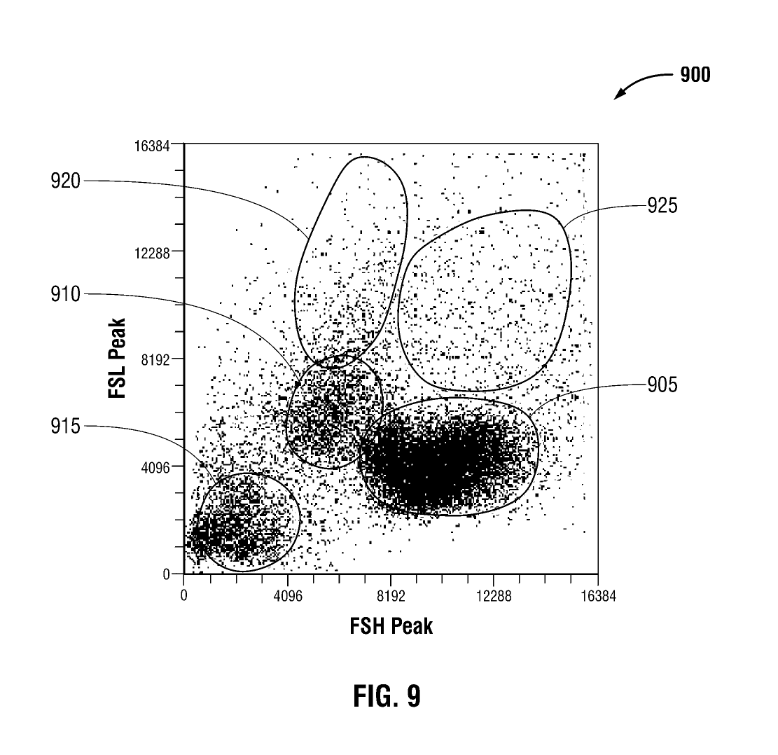

As stated above, there are many reasons for lymphadenopathy. For example, autoimmune disease, bacterial or viral infection, pyogranulomatous, or metastatic mast cell tumor. FIG. 9 is a scatter profile 900 of reactive inflammation in a mammal. Scatter profile 900 includes red blood cell region 905. The increased red cell contamination is not unexpected given the inflamed and reactive lymph node. The non-intact cells and debris are located in region 915. The normal lymphocyte region 910 is consistent with the normal lymph node aspirate region 610 in FIG. 6. Note the absence of cells in region 920, where enlarged and abnormal lymphocytes would typically be detected. Instead region 925 (about 12,000 FSH peak and about 10,000 FSL peak) contains a population of cells which represent granulocytes and monocytes as characterized by the greater peak FSH. The presence of granulocytes and monocytes are hallmarks of reactive inflammation. Accordingly, a veterinarian or technician may conclude that there is some degree of granulocytic or monocytic inflammation responsible for the lymphadenopathy.

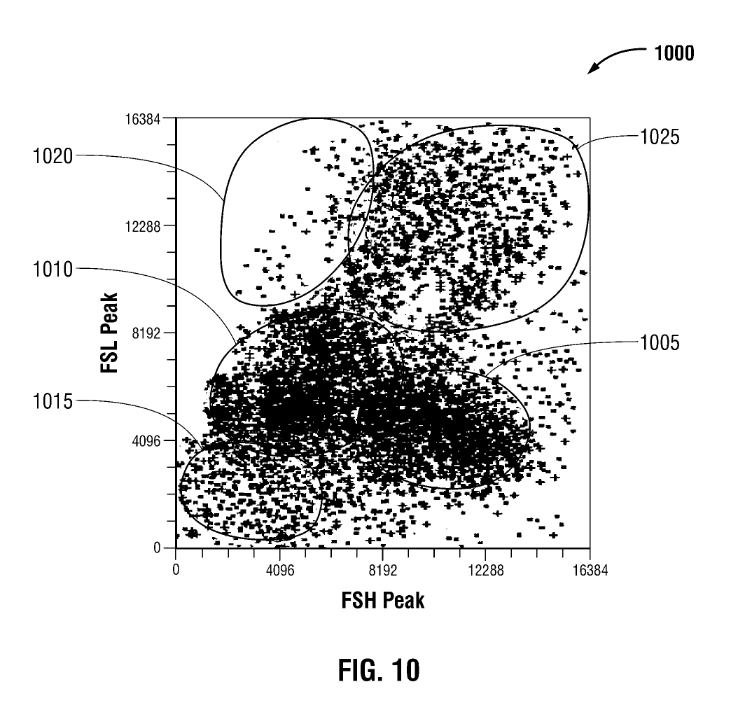

FIG. 10 is a scatter profile 1000 of a metastatic mast cell tumor. Mast cells, similar to granulocytes, are very granular. Mast cell tumors can lead to either elevated and suppressed levels of white blood cells. The scatter profile 1000 includes a red blood cell region 1005 and a non-intact cells and debris region 1015. The normal lymphocyte region 1010 is similar to the normal lymph node aspirate region 610 in FIG. 6. Note the absence of cells in region 1020, where enlarged and abnormal lymphocytes would be found. Instead region 1025 (about 12,000 FSL peak and about 11,000 FSH peak) contains a population of cells which represent metastatic mast cells and potentially other granulocytes as characterized by the greater peak FSH. As a result, a veterinarian or technician may conclude that scatter profile 1000 is likely due to metastatic mast cells or inflammation, rather than lymphoma.

The values of FSH peak and FSL peak described above for the various regions are approximate values to describe the general area.

While the above description contains many specifics, these specifics should not be construed as limitations on the scope of the present disclosure, but merely as exemplifications of preferred embodiments thereof.

For example, although the present disclosure specifies a method for identifying and determining severity or remission of disease states based on white blood cell subpopulations from canine lymph node aspirate, it is not so limited, but rather can be utilized for any lymph node aspirate sample using flow cytometer where disease may be present. Those skilled in the art will envision many other possible variations that are within the scope and spirit of the present disclosure.

What is claimed is:

1. A method for identifying a cause of lymphadenopathy in a mammal comprising:
collecting lymph node aspirate from a mammal;
scanning the lymph node aspirate in a flow cytometer to generate a diagnostic scan;
comparing the diagnostic scan to a known normal scan;
identifying differences between the diagnostic scan and the known normal scan; and
identifying similarities between the diagnostic scan and known disease scans to identify a cause of lymphadenopathy.

2. The method according to claim 1, wherein the known disease scan is selected from the group consisting of a lymphoma scan, a mast cell tumor scan, a reactive inflammation scan and combinations thereof.

3. The method according to claim 1, wherein the scanning step is performed using a multi-angle scattered flow cytometer.

4. The method according to claim 1, wherein the diagnostic scan, known normal scan, and known disease scan are scatter profiles.

5. The method according to claim 1, wherein the mammal is a canine.

6. The method according to claim 1, wherein the mammal is a feline.

7. The method according to claim 1, wherein the scanning step includes scanning the lymph node aspirate in the absence of a marker.

8. The method according to claim 1, further comprising suspending the lymph node aspirate in a saline buffer in the absence of a fluorescent dye.

9. The method according to claim 1, further comprising storing the diagnostic scan, known normal scan, and known disease scan in a storage database.

10. The method according to claim 1, wherein the flow cytometer uses high forward scattered light and low forward scattered light.

11. A method of monitoring treatment of a diseased mammal comprising:
aspirating a lymph node of a diseased mammal to obtain pre-treatment aspirate;
scanning the pre-treatment aspirate using a multi-angle scattered flow cytometer to generate a first scatter profile,
aspirating a lymph node of a diseased mammal following treatment to obtain post-treatment aspirate;
scanning the post-treatment aspirate aspirated following treatment using the multi-angle scattered flow cytometer to generate a second scatter profile, and
identifying differences between the first scatter profile and the second scatter profile.

12. The method according to claim 11, wherein the pre-treatment aspirate and the post-treatment aspirate comprise a lymphocyte subpopulation.

13. The method according to claim 12, wherein the lymphocyte subpopulation is selected from the group consisting of normal lymphocytes, abnormal lymphocytes, granulocytes, monocytes, metastatic mast cells, T-cells, B-cells and combinations thereof.

14. The method according to claim 12, wherein the first scatter profile and the second scatter profile represent lymphocyte subpopulations.

15. The method according to claim 13, wherein the lymphocytes are B-cells.

16. The method according to claim 13, wherein the lymphocytes are T-cells

17. The method according to claim 12, wherein the step of identifying further comprises quantifying the lymphocyte subpopulation to determine the effectiveness of the treatment.

18. The method according to claim 12, wherein the first and second scatter profiles represent a size of the lymphocyte subpopulation.

19. The method according to claim 11, further comprising comparing the first scatter profile to a baseline scatter profile.

20. The method according to claim 19, wherein the baseline scatter profile represents healthy mammal aspirate.

21. The method according to claim 11, wherein the step of scanning the pre-treatment aspirate further comprises high forward scattered light and low forward scattered light.

22. The method according to claim 11, wherein the step of scanning the post-treatment aspirate further comprises high forward scattered light and low forward scattered light.

23. The method according to claim 11, wherein the step of scanning the pre-treatment aspirate further comprises:
diluting the pre-treatment aspirate in a buffer; and
flowing the dilute pre-treatment aspirate through a flow cytometer.

24. The method according to claim 11, wherein the step of scanning the post-treatment aspirate further comprises:
diluting the post-treatment aspirate in a buffer; and
flowing the dilute post-treatment aspirate through a flow cytometer.

25. The method according to claim 11, further comprising suspending the pre-treatment aspirate in a buffer in the absence of a fluorescent dye.

26. The method according to claim 11, further comprising suspending the post-treatment aspirate in a buffer in the absence of a fluorescent dye.

27. The method according to claim 11 further comprising the steps of:
aspirating a lymph node of the diseased mammal following additional treatment to obtain additional treatment aspirate;
scanning the additional treatment aspirate using a multi-angle scattered flow cytometer to generate an additional scatter profile; and
identifying differences between the first scatter profile, second scatter profile, and additional scatter profile.

28. The method according to claim 26, wherein the step of identifying differences between the first scatter profile, second scatter profile, and additional scatter profile comprises quantifying a lymphocyte subpopulation to determine the effectiveness of the additional treatment.

29. The method according to claim 27, wherein the additional treatment is the same as the treatment.

30. The method according to claim 27, wherein the additional treatment is different than the treatment.

31. A method of assessing disease state comprising
collecting lymph node aspirate from a mammal;
scanning the lymph node aspirate in a flow cytometer to generate a first disease state scan;
collecting an additional lymph node aspirate from the mammal;
scanning the additional lymph node aspirate in a flow cytometer to generate an additional disease state scan; and
comparing the first disease state scan to the additional disease state scan to assess the disease state.

32. The method according to claim 31 wherein the first diagnostic scan and the additional diagnostic scan comprise lymphocyte subpopulations.

33. The method according to claim 32, wherein the lymphocyte subpopulation is selected from the group consisting of normal lymphocytes, abnormal lymphocytes, granulocytes, monocytes, metastatic mast cells, T-cells, B-cells and combinations thereof.

34. The method according to claim 32, wherein the lymphocyte subpopulation comprises normal lymphocytes.

35. The method according to claim 34, wherein the additional diagnostic scan comprises a greater quantity of normal lymphocytes than the first diagnostic scan.
